(19) United States
(12) Patent Application Publication
(10) Pub. No.: US 2004/0137538 A1
(43) Pub. Date: Jul. 15, 2004

(54) Cancer comprehensive assay kit for identifying cancer protein patterns

(76) Inventor: Sherry A. Bradford, Grand Island, NY (US)

Correspondence Address: Walter W. Duft, Suite 10, 10255 Main Street, Clarence, NY, 14031, US

(21) Appl. No.: 10/340,549

(22) Filed: Jan. 10, 2003

(51) Int. Cl. G01N 33/574

(52) U.S. Cl. 435/7.23; 422/61

(57) ABSTRACT

A cancer therapy comprehensive assay kit for characterizing a cancer tumor for medical diagnosis and treatment. The assay kit facilitates determination of a cancer protein pattern based on detected levels of biomolecular markers (BMMs) associated with a patient's tumor. A cancer therapy regimen is selected based on the cancer protein pattern for eradicating the tumor.

### BACKGROUND OF THE INVENTION

1. Field of the Invention

The present invention relates to the detection and treatment of cancer. More particularly, the invention concerns a comprehensive assay kit for identifying a cancer protein pattern and determining a course of chemotherapy and/or radiotherapy.

2. Description of Prior Art

By way of background, cancer patients are generally treated by standard and generic protocols, with the type of protocol being largely determined according to the tumor's generic histologically determined stage (determined through biopsy and tumor marker testing), and the individual clinician's experience and preference. This form of treatment is based on statistical information derived from historical data and is not individualized to the specific patient. Based on microscopic examination, tumors of the same type appear very similar. However, tumors within a given patient may demonstrate divergent growth curves and characteristics as well as disparate responses to chemoregimens due to biochemical and genetic nonequivalence. Thus, it cannot be said that every and all patients exhibiting the identical microscopic narrative, and hence the same stage, will respond favorably to the exact same empiric “cure-one-cure-all” therapy.

In an effort to individualize cancer therapy, a clinician may have in-vitro testing performed to pre-determine the effects of chemotherapeutic agents on tumor cells obtained from the patient. According to the usual technique, patient tumor cells are allowed to grow and then tested only for resistance to cancer treatment drugs. A drug determined to be ineffective relative to the in-vitro testing may then be eliminated as the drug of choice for the patient.

There are multiple reasons why this approach may not be effective: First, because the tested tumors are grown in a culture, they represent a homogenous cell population. The patient's actual tumor is typically composed of multiple diverse cell populations in varying stages of cell cycle, and expressing various extracellular, cytoplasmic, and nuclear antigens in varying concentrations, as well as containing normal stromal cells, epithelial populations and vascular endothelial cell populations. Second, by the time the in-vitro tumor has been grown out and tested, first line chemotherapy cannot be realized due to the time needed for cellular growth (assuming the tumor grows at all). This mandates second line regimes. Moreover, when the tumor is exposed to a first line regimen that, may not work, the tumor is given enough time to assemble a “blue print” in which to manufacture multi-drug resistance proteins to fight any drug regimen to which it may be subsequently exposed. Third, the drugs tested in-vitro are used at overtly high concentrations that are not physiologically achievable in-vivo. Unfortunately, the use of higher than peak plasma concentrations of drug can overwhelm the cell's infrastructure. This may “confuse” a cancer cell so that it doesn't know whether to obey its innate signal to thrive and grow or obey the extra cellular drug signal to cease growth and die. Thus, the cell merely waits for a ratiocinate signal., By the time this equilibrium is reached, the body has excreted the drug and the cell “awakens” to follow its innate signal to thrive and grow. Moreover, this “conditioning” has now allowed the cell to manufacture weapons to fight the next round of death signals (drugs). As indicated above, such weapons include multi-drug resistant proteins that pump the drug out of its intracellular milieu and into the external environment. Thus, the cell becomes drug savvy and therefore impervious to the assault. Fourth, individualized in-vitro testing is premised on the use of a single chemotherapeutic agent and is unable to evaluate the effects of combinations of agents. Applicant submits that a multi-parametered tumor must be combated with a multiplicity of agents if the tumor is to be eradicated.

Accordingly, an improved assay kit for cancer chemotherapy (and radiotherapy) is needed. What is particularly required is an assay technique that is specific to individual cancer patients and considers the gross tumor cellular content as well as molecules that characterize the tumor milieu, thereby allowing a patient's progress to be followed and ensuring that the therapy is or is not efficacious.

### SUMMARY OF THE INVENTION

The foregoing problem is solved and an advance in the art is provided by a novel cancer comprehensive assay kit in which oncolytic product selection and dosing (as well as other therapies) are determined through a single test to identify a patient's individualized, cancer protein pattern of physiologically present biomolecular markers and the up or down regulation of these markers from basal levels thereof. In preferred implementations of the invention, the assay kit includes a frame structure and a plurality of test wells associated with the frame structure. The test wells are arranged to form plural test well rows and plural test well columns. Each test well has a surface configuration that is coated with a capture protein. The capture proteins are specific to multiple biomolecular markers (BMMs) and are arranged such that capture proteins specific to a particular BMM are associated with a single test well column. A set of detection proteins is provided for use with one of the test well columns. Each detection protein is specific to the same BMM as the capture protein associated with the same test well column. At least some of the test wells of each test well column are adapted to receive assay evaluation samples obtained from a patient tumor sample or from a patient serum/plasma sample. The BMMs to which the capture proteins and the detection proteins are specific are selected so that the test well columns may be used to collectively test for a cancer protein pattern based on detected levels of multiple biomolecular markers (BMMs) associated with a patient's tumor, and so that a cancer therapy regimen may be selected based on said cancer protein pattern for eradicating the tumor.

It is therefore an object of the invention to target cancer therapy to a specific cancer patient so that the patient's tumor is not exposed to an inappropriate regimen of drugs, thereby increasing efficacy.

Another object of the invention is to examine the heterogeneity of an entire tumor, thereby taking into consideration every cell that composes the tumor and not just those that are in DNA synthesis.

A further object of the invention is to evaluate an individual cancer patient and not use a generic treatment that is empirically and generically chosen merely based on staging for a specific cancer.

A further object of the invention is to target first-line chemotherapy.

A further object of the invention is to predetermine if radiotherapy will be effective, partially effective or not effective at all in cancer patients. This rationale is based on the fact that, like chemotherapy, radiotherapy is also chosen based on morphological characteristics and not individualized based on the specific patient's tumor heterogenic cell population characteristics.

A further object of the invention is to be able to follow and monitor a specific patient to ensure that chemotherapy or radiotherapy has been efficacious.

A further object of the invention is to be able to determine if previously treated patient in remission is at risk for recurrence, relapse or metastasis.

A further object of the invention is to be able to screen for the possible onset of cancer using the disclosed methodology during routine physical examination.

### BRIEF DESCRIPTION OF THE DRAWINGS

The foregoing and other features and advantages of the invention will be apparent from the following more particular description of preferred embodiments of the invention, as illustrated in the accompanying Drawings in which:

FIG. 1 is a plan view of an exemplary assay kit for use in accordance with the invention;

FIGS. 2A-2F are diagrammatic views showing exemplary assay steps performed in accordance with the invention; and

FIG. 3 is a diagrammatic plan view of showing how individual test wells may be used in the assay kit of FIG. 1.

### DETAILED DESCRIPTION OF PREFERRED EMBODIMENTS

Applicant has observed that cancer treatment evaluation must be individualized based on the patient's heterogeneous tumor cell populations. A course of treatment cannot be determined merely by morphological characteristics (staging) alone insofar as the biochemical and genetic parameters are not reflected morphologically. The invention thus proposes that cancer therapy be based on tumor biomolecular (biochemical/genetic) characteristics and not merely on staging. This is accomplished by evaluating the totality of a patient's tumor cell populations (without having to grow out a tumor in-vitro) based on a plurality of the specific individual's tumor parameters to determine the chemotherapy and/or radiotherapy regimen needed to eradicate the entire tumor mass. This evaluation is performed within the time constraints necessary for targeting first line treatment regimens, thereby lessening the chance that any cells will escape the “combatant” regimen while realizing few or no side effects by the patient.

The assay kit of the invention can realize results within 24-48 hours. The assay kit is adapted so that a biomolecular profile is performed relative to a patient's own cancer protein pattern of biomolecular markers (BMMs). The BMMs can be antigens or antibodies (proteins), such as specific tumor receptors, growth factor receptors, basement membrane components, adhesion molecules or angiogenesis components. One example is VEGF (vascular endothelial growth factor) receptor. An adult normally never vascularizes unless there is a pathological condition. This could include wound healing and in the female, normal menses or pregnancy, but is also associated with a growing tumor. To progress beyond 3 mm in size, a tumor must become invested with vessels in order to get rid of toxins and take in nutrients. The tumor will thus have an abundance of VEGF receptors so that it can derive stimulus from growth factor molecules in the circulating blood.

More generally, the assay kit of the invention evaluates two classes of BMMs associated with cancer patients. The first BMM class consists of proteins (Class I BMMs) that can be targeted for treatment by way of modulating drugs that regulate (e.g., “cap”) the targeted protein (e.g., signal transduction pathway (STP) monoclonal antibody drugs). Exemplary Class I BMMs include estrogen, receptors (ER), progesterone receptors (PR), androgen receptors (AR), and epidermal growth factor (EGFR). The second BMM class consists of proteins (Class II BMMs) that provide information about a patient's overall cancer process, such as tumor markers that may indicate cancer onset, progression and regression. Examples include cancer antigen 125 (CA-125), cancer antigen 19.9 (CA19.9), CU-18 breast related antigen, S-100, DF-3 blood factor, tumor suppressor protein p53 and c-myc oncogene. Note that some proteins fall into both classes. Examples include Her2/neu growth factor receptors, multidrug resistance proteins (MRP), lung resistance proteins (LRP), proliferating cell nuclear antigen (PCNA) and urokinase plasminogen activator (uPA).

Procedure

Initially, a tumor sample is obtained from the patient and homogenated into a liquefied state. The homogenate of the solid tumor will contain the cellular components that can be retrieved and used (with dilution) as an assay evaluation sample. If needed, the assay evaluation sample can be further diluted to allow evaluation of a multiplicity of BMMs (merely multiply the obtained result by the dilution factor to obtain the actual result). Blood serum/plasma may also be used to provide the assay evaluation sample insofar as the circulatory system contains proteins shed by the solid tumor. Alternatively, other body fluids, such as saliva, could be obtained from the patient to provide the assay evaluation sample. The

The assay evaluation sample is tagged with labeled detection antibodies or antigens that have been fluorinated or otherwise rendered detectable. Each detection antibody/antigen is selected to bind to a selected Class I or Class II BMM that is considered indicative of a characteristic of the patient's tumor, with the Class I BMMs targeting proteins treatable with modulating drugs, and the Class II BNMs providing process information such as the type of cancer, the tumor's growth stage, and the tumor's ability to resist certain chemotherapies or radiotherapies. The detection antibodies/antigens will preferably be labeled for use with an assay methodology such as ELISA (Enzyme-Linked Immunosorbent Assay) in which fluorescence is used to detect the presence of the labeled material and thus the BMM to which it is bound. Alternatively, the detection antibodies/antigens could be labeled for detection using the laser photometrics of a flow cytometer. In addition to the detection antibodies/antigens, capture antigens/antibodies specific to the BMMs of interest are used to provide a sandwich assay format. The capture antibodies/antigens allow the BMMs to be bound to a microtiter plate or other carrier for handling.

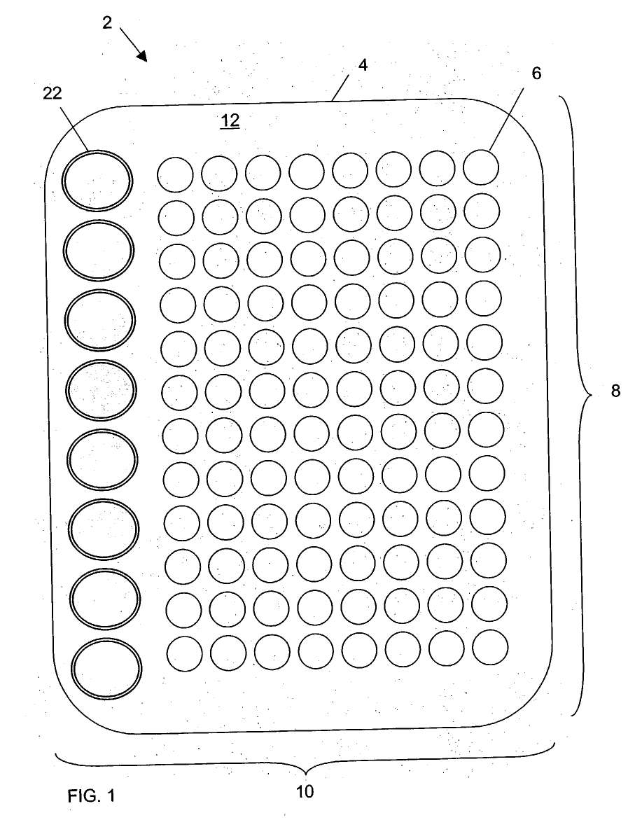

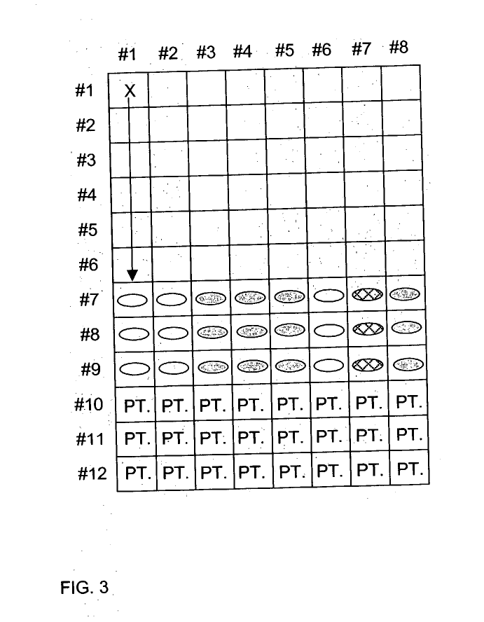

In a preferred embodiment of the invention, and as shown in FIG. 1, a multiple test well assay kit 2 is provided to simultaneously test for a cancer protein pattern comprising a plurality of BMMs using ELISA evaluation. The test kit 2 is constructed using a commercially available microtiter plate 4 having an array of test wells. FIG. 1 shows a microtiter plate configured in a 96 well format, but smaller or larger sizes could be used depending on the number of BMMs to be evaluated. In the 96 well size, there are 96 separate test wells 6 arranged to provide a two dimensional array comprised of well rows 8 and well columns 10. The microtiter plate 4 is made from inert plastic or other suitable material. It can be molded as a single structure in which the test wells 6 are integrally formed together in conjunction with a surrounding frame 12. Alternatively, a strip well construction can be used in which the frame 12 is separately constructed from the test wells 6 so that the test wells can be removed from the frame. The test wells 6 that define each separate well row 8, or each separate well column 10, can then be joined together to facilitate insertion in and removal from the frame 12 as a group. If desired, the test wells 6 that comprise each well row 8 or well column 10 can be joined to each other by breakable connections so that individual test wells can be separated from the well row or well column. As described in more detail below in connection with FIG. 3, if the test wells 6 of each well column 10 are joined together, each well column 10 can be assigned for use in identifying a particular BMM of interest. Then, if the clinician does not want to look at that particular BMM, the well column 10 for that BMM can then be stripped out of the microtiter plate 4. A pertinent marker strip may be substituted if desired.

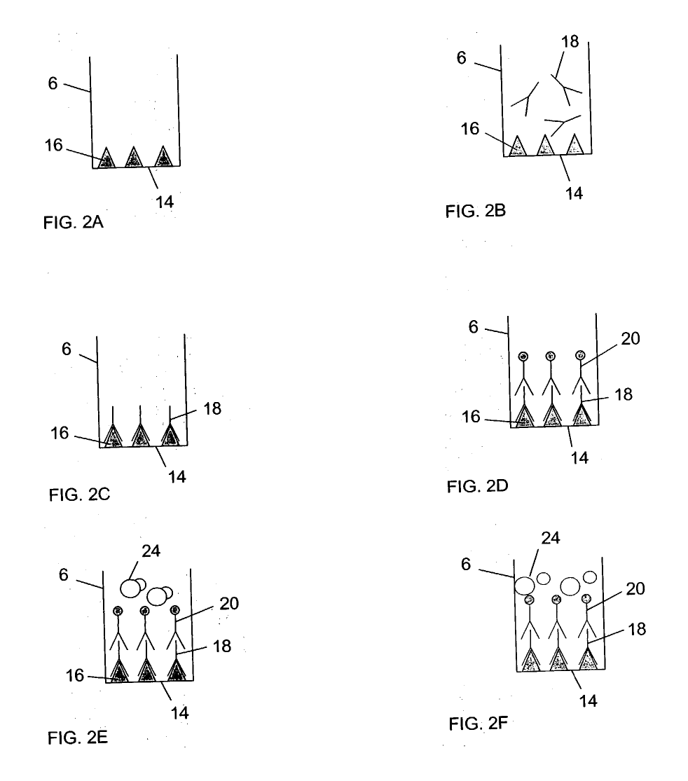

Turning now to FIGS. 2A-2F, each test well 6 has a bottom surface configuration 14 that is conventionally coated with capture antigen or antibody material 16 to provide a solid phase membrane for binding target BMMs in the patient's assay evaluation sample. As is generally known, the antigen/antibody material 16 can be coated on the bottom surface 14 using a coating buffer that enhances binding. Sites that are unoccupied by the capture antigen or antibody material 16 may be blocked with a blocking buffer to prevent non-specific binding of proteins in the assay evaluation sample, if so desired. FIG. 2A shows a test well 6 that is constructed in the foregoing manner and ready to receive an assay evaluation sample. FIG. 2B shows the same test well 6 after an assay evaluation sample obtained from a patient is placed in the well. The assay evaluation sample is assumed to contain BMMs 18 that are specific to the capture antigens or antibody material 16 bound to the well's bottom surface configuration 14. In FIG. 2C, the BMMs 18 are shown after they bind to the antigen or antibody material 16. Non-specific proteins that do not bind to the antigen or antibody material 16 are washed away. In FIG. 2D, enzyme labeled (e.g., horseradish peroxidase) detection antibodies or antigens 20 are added to the test well 6, where they bind, to the captured BMMs 18. Unbound detection antibodies/antigens 20 are washed away. The detection antibodies/antigens 20 can be provided in individual vials 22 as part of the kit 2. The vials 22 may be carried on the frame 12, or separately provided in the packaging for the kit 2. In FIG. 2E, a colorimetric substrate 24 (e.g., o-phenylenediamine dihydrochloride, tetramethylbenzidine (TMB)) is added to the test well 6. In FIG. 2F, the enzymes on the detection antibodies/antigens 20 cleave the substrate 24, causing a color change of the substrate solution. The intensity of the color (absorbance) is quantified using a spectrophotometer (e.g., ELISA reader) and is proportional to the number of target proteins in the assay evaluation sample.

As shown diagrammatically in FIG. 3, the test kit 2 is preferably configured to evaluate several BMMs in a single test, with each well column 10 being assigned to a particular BMM. In FIG. 3, there are eight well columns 10 labeled #1 through #8. Thus, eight BMMs may be tested. There are also twelve rows labeled #1 through #12. Rows #1 through #6 are used to provide standard curves to facilitate evaluation and retrieval of data results for particular BMMs. Each well in rows #1 through #6 thus contains a sample of a BMM of interest at an established concentration. The measured absorbance obtained for each BMM sample in rows #1 through #6 is used to define a standard curve for each BMM that correlates absorbance with BMM concentration. Rows #7 through #9 are used to provide three different control levels, low, medium and high of the BMMs of interest. The control samples of rows #7 through #9 indicate that the assay test is functioning correctly and that patient results will be valid. Rows #10 through #12 are used for the patient's assay evaluation samples. Three rows of samples are tested and the mean test result values are used to assure accuracy. The various controls are assigned a specific concentration along with a standard deviation (+/−). If results fall within the designated assigned values associated with the standard curves and controls, then this indicates the curves were set up correctly and the patient results are valid.

The results of the assay test can be used to determine a course of treatment to administer to the patient. The overall methodology is to identify a cancer protein pattern of Class I BMMs based on the detected levels of these proteins. The Class I BMMs will generally be either tumor promoting proteins or tumor suppressor proteins. The assay test will identify the extent to which any tumor promoting proteins are upregulated and/or any tumor promoting proteins are downregulated. From this pattern, and with the assistance of information provided by the presence or absence of the Class II BMMs, a chemo-regimen or radio-regimen may be targeted to maximize the eradication of the patient's, solid tumor.

Most important are the Class I BMMs because they signify the presence of proteins that can be modulated by conventional STP drugs. Unlike current treatments in which one or more of such drugs are prescribed based on tumor staging, the drugs are selectively combined into a chemo-suite that directly corresponds to a specific patient's BMM pattern revealed for that patient by the assay test. The treatment is thus customized to target cells that express the BMMs represented in the pattern. The significance of the Class II BMMs can be appreciated from the fact that each of the Class I BMMs is a normally expressed antigen that may be found in non-cancerous tissue at basal levels. Even if a particular Class I BMM is above or below its basal level, it may not be appropriate to make a diagnosis of cancer. For example, most individuals do not normally express up-regulated levels of VEGF. However, as previously mentioned, an assay test of a female during normal menses or pregnancy could reveal such up-regulation. On the other hand, the additional presence of a Class II BMM such as CA-125 could lead to a different diagnosis. Similarly, elevated levels of more than one tumor promoting protein or decreased levels of more than one tumor suppressor protein could provide a more definitive diagnosis. For example, the presence of two Class I BMMs would likely be interpreted as a pre-cancerous condition. The presence of three or more Class I BMMs would likely be interpreted as cancer.

Advantageously, the assay kit of the invention facilitates such definitive diagnoses by testing for the patient's cancer protein patterns rather than individual proteins, such as various prior art assays that identify individual tumor markers. This is particularly useful for first line chemotherapy. Rather than prescribing drugs according conventional staging methods and running the risk that the drugs will be inefficacious and promote drug resistance that impacts second line treatment, a carefully targeted treatment suite can be prescribed that the practitioner reasonably knows will control the identified BMMs.

Exemplary Assay Kits

A number of basic assay kit profiles have been developed to characterize different cancers. These profiles variously target tumor cell proliferation, proliferation signal-transduction pathways, growth factors, growth factor receptors, oncogenes, tumor suppressor genes, multi-drug resistance, angiogenesis, invasion/metastasis, apoptosis, hormone receptors, WBC infiltration, non-specific tumor markers, organ-specific tumor markers, extracellular matrix proteins, adhesion proteins, and proteins involved with DNA and DNA repair.

Table 1 below illustrates several exemplary profiles that respectively characterize ovarian cancer, ovarian/peritoneal cancer, and ovarian/gall bladder/peritoneal cancer. It will be seen that either a basic or comprehensive profile may be used for each cancer. A basic profile may comprise a gradient either greater than or equal to five BMMs. A comprehensive profile may comprise a gradient greater than or equal to ten BMMs. In Table 1 below, three exemplary basic profiles and three exemplary comprehensive profiles are shown. The first two profiles are for ovarian cancer, the second two are for ovarian/peritoneal cancer, and the third two profiles are for ovarian/gall bladder/peritoneal cancer.

TABLE 1

| TUMOR TYPE | BASIC PROFILE | COMP. PROFILE |
|---|---|---|
| OVARIAN | ER/PR, Her2/neu, MRP, | ER/PR/AR, Her2/neu, MRP, |
| | LRP, EGFR | LRP, EGFR, CA-125, CU- |
| | | 18, PCNA, DF 3, uPA |
| OVARIAN/PERITONEAL | S-100, PCNA, MDR-1, | S-100, PCNA, MDR-1, |
| | EGFR, ER/PR/AR | EGFR, ER/PR/AR, Ki-67, |
| | | p53, Her2/neu, MRP, LRP, |
| | | EGFR, CA-125, uPA |
| OVARIAN/GALLBLADDER/ | S-100, PCNA, MDR-1, | S-100, PCNA, MDR-1, |
| PERITONEAL | EGFR ER/PR/AR, PP, p53, | EGFR ER/PR/AR, PP, MRP, |
| | c-myc | S-100, NSE, LMW Keratin, |
| | | p53, TS, CD43, CEA, CD31, |
| | | CA 242, c-myc, PDECGF, |
| | | VIP |

Ovarian Cancer

The ovarian basic profile includes antibodies to detect for the presence of estrogen receptors (ER), progesterone receptors (PR), Her2/neu growth factor receptors, multidrug resistance proteins (MRP), lung drug resistance proteins (LRP) and epidermal growth factor receptors (EGFR). The ovarian comprehensive profile includes the same markers plus markers to detect for the presence of androgen receptors (AR), CA-125 antigen, CU-18 breast-related antigen, proliferating cell nuclear antigen (PCNA), DF-3 blood factor and urokinase plasminogen activator (uPA).

The capture antibodies that may be used to detect the above-identified ovarian cancer BMMs are set forth in Table 2 below. They are all conventionally available monoclonal or polyclonal antibodies with polyclonal antibodies being preferred to ensure detection of the specific proteins of interest. These proteins will be composed of multiple epitopes to which the polyclonal antibodies may bind. Monoclonal antibodies will target only one epitope and if that epitope has mutated, the monoclonal antibody will not bind. The assay would then give a false indication that the protein of interest is not present when in fact it is. Because a polyclonal antibody targets many epitopes on the protein of interest, there is an increased chance that the protein will be detected by the assay.

TABLE 2

| BMM | CAPTURE ANTIBODY |
|---|---|
| ER/PR/AR | ER/PR/AR antibody |
| Her2/neu | Her2/neu antibody |
| MRP | MRP antibody |
| LRP | LRP antibody |
| EGFR | EGFR antibody |
| CA-125 | CA-125 antibody |
| CU-18 | CU-18 antibody |
| PCNA | PCNA antibody |
| DF-3 | DF-3 antibody |
| UPA | uPA antibody |

Note that all of the above ovarian cancer BMMs except CA-125, CU-18 and DF 3 may be considered Class I BMMs. All of the BMMs except ER/PR and EGFR may also be considered Class II BMMs. Relative to the BMMs having Class I status, Table 3 below lists conventional drugs that may be used to modulate such proteins:

TABLE 3

| Class One BMM | Drug |
|---|---|
| ER/PR/AR | Hormone capping antibodies |
| Her/neu | Herceptin |
| MRP | Glucosylceramide synthase antisense cDNA |
| LRP | Clafazimine |
| EGFR | ZD 1839 or vaccine |
| PCNA | NAMI-A (Ruthenium Complex) |
| UPA | WX-360 (uPAR-antagonist) |

Ovarian/Peritoneal Cancer

The ovarian/peritoneal basic profile includes markers to detect for the presence of cancer antigen 19-9 (C Al 9-9), S-100, proliferating cell nuclear antigen (PCNA), multidrug resistance-1 (MDR-1), epidermal growth factor receptors (EGFR), estrogen receptors (ER), progesterone receptors (PR) and androgen receptors (AR). The ovarian/peritoneal comprehensive profile includes the same markers plus markers to detect for the presence of monoclonal antibody Ki-67, tumor suppressor protein (p53), Her2/neu growth factor receptors, multidrug resistance proteins (MRP), lung drug resistance proteins (LRP), cancer antigen 125 (CA125) and urokinase plasminogen activator (uPA).

The capture antibodies that may be used to detect the above-identified ovarian/peritoneal cancer BMMs are set forth in Table 4 below. They are all conventionally available polyclonal or monoclonal antibodies (with polyclonal antibodies being preferred), as follows:

TABLE 4

| BMM | CAPTURE ANTIBODY |
|---|---|
| CA19-9 | CA19-9 antibody |
| S-100 | S-100 antibody |
| PCNA | PCNA antibody |
| MDR-1 | MDR-1 antibody |
| EGFR | EGFR antibody |
| ER/PR/AR | ER/PR/AR antibody |
| Ki-67 | Ki-67 antibody |
| p53 | p53 antibody |
| Her2/neu | Her2/neu antibody |
| MRP | MRP antibody |
| LRP | LRP antibody |
| CA-125 | CA-125 antibody |
| UPA | uPA antibody |

Note that all of the above ovarian/peritoneal cancer BMMs except CA-19-9, S-100, p53 and CA-125 may be considered Class I BMMs. All of the BMMs except ER/PR/AR and EGFR may also be considered Class II BMMs. Relative to the BMMs having Class I status, Table 5 below lists conventional drugs that may be used to modulate such proteins:

TABLE 5

| Class One BMM | Drug |
|---|---|
| ER/PR/AR | Hormone capping antibodies |
| Her/neu | Herceptin |
| MRP | Glucosylceramide synthase antisense cDNA |
| LRP | Clafazimine |
| EGFR | ZD 1839 or vaccine |
| MDR-1 | Taxanes |
| Ki-67 | S-phase targeting drugs |
| PCNA | NAMI-A (Ruthenium Complex) |
| UPA | WX-360 (uPAR-antagonist) |

Ovarian/Gall Bladder/Peritoneal Cancer

The ovarian/gall bladder/peritoneal basic profile includes markers to detect for the presence of cancer antigen 19-9 (CA19-9), S-100, proliferating cell nuclear antigen (PCNA), MDR-1, epidermal growth factor receptors (EGFR), estrogen receptors (ER), progesterone receptors (PR), androgen receptors (AR), PP, tumor suppressor protein (p53) and c-myc. The ovarian/gall bladder/peritoneal comprehensive profile includes the same markers plus markers to detect for the presence of MRP, neuron-specific enolase (NSE), LMW Keratin, thymidylate synthase (TS), sialophorin (CD43), carcinoembryonic antigen (CEA), PECAM-1 (CD31), cancer antigen 242 (CA242), platelet-derived endothelial cell growth factor (PDECGF) and vasoactive intestinal peptide (VIP).

The antibodies/antigens that may be used to detect the above-identified ovarian/gall bladder/peritoneal cancer BMMs are set forth in Table 6 below. They are all conventionally available polyclonal or monoclonal antibodies (with polyclonal antibodies being preferred), as follows:

TABLE 6

| BMM | CAPTURE ANTIBODY |
|---|---|
| CA19-9 | CA19-9 antibody |
| S-100 | S-100 antibody |
| PCNA | PCNA antibody |
| MDR-1 | MDR-1 antibody |
| EGFR | EGFR antibody |
| ER/PR/AR | ER/PR/AR antibody |
| PP | PP antibody |
| p53 | p53 antibody |
| c-myc | c-myc antibody |
| MRP | MRP antibody |
| NSE | NSE antibody |
| LMW Keratin | LMW keratin antibody |
| TS | TS antibody |
| CD43 | CD43 antibody |
| CEA | CEA antibody |
| CD31 | CD31 antibody |
| CA 242 | CA 242 antibody |
| PDECGF | PDECGF antibody |
| VIP | VIP polyclonal antibody |

Note that all of the above ovarian/peritoneal/gall bladder cancer BMMs except CA-19-9, S-100, p53, c-myc and CA 242 may be considered Class I BMMs. All of the BMMs except ER/PR/AR and EGFR may also be considered Class II BMMs. Relative to the BMMs having Class I status, Table 7 below lists conventional drugs may be used to modulate such proteins:

TABLE 7

| Class One BMM | Drug |
|---|---|
| ER/PR/AR | Hormone capping antibodies |
| Her/neu | Herceptin |
| MRP | Glucosylceramide synthase antisense cDNA |
| LRP | Clafazimine |
| EGFR | ZD 1839 or vaccine |
| PCNA | NAMI-A (Ruthenium Complex) |
| MDR-1 | Taxanes |
| PP | Liposomal daunorubicin antisense cDNA |
| NSE | Cyclophosphamide, Etopaside, Soxorubicin |
| LMW Keratin | LMW Keratin (cytoKeratin) capping antibody |
| TS | Fluoropyrimidines (5-FU) |
| CD43 | Anti CD43 |
| CEA | Prodrug genetherapy METgene-SeMET |
| CD31 | Anti CD31 |

Additional Profiles and Panels

Many other exemplary assay kit profiles and panels can be constructed in accordance with the present invention. Table 8 below shows a number of additional assay kit profiles, while Table 9 below shows a number of smaller assay kit panels for targeting specific protein groups. As explained below, many of the panels of Table 9 can be used to augment the profiles of Table 8, thereby providing additional information about patient treatment options.

TABLE 8

| TUMOR TYPE | BASIC PROFILE | COMP. PROFILE |
|---|---|---|
| Adeno-Carcinoma | ACTH, B72.3, BCA225, Bcl-2, | ACTH, B72.3, BCA225, Bcl-2, |
| | CA15.3 | CA15.3, CA125, CEA/D-14, |
| | | CyclinD1, PCNA, Ki-67, MLRP, |
| | | MDR-1 (ψ) (χ) (f) (λ) |
| Bladder | p53, Her2/neu (p185), PCNA, | p53, Her2/neu (p185), PCNA, MDR- |
| | MDR-1, EGFR, | 1, EGFR, Ki-67, pan-ras, Bcl-2, Bcl-x, |
| | | Rb (π) |
| Brain | p53, Her2/neu, MGMT, Ki-67, | p53, Her2/neu, MGMT, Ki-67, MDR- |
| | MDR-1, GFAP, Syn | 1, GFAP, Syn, CD35, CD31, PCNA, |
| | | VEGFR, PDGFR |
| | | (ψ) (♥) (∞) |
| Breast [Adeno- | ER/PR, Her2/neu, TS, BCA-125, | ER/PR, Her2/neu, TS, BCA-125, |
| Carcinomas] | MDR-1, MRP | MDR-1, MRP, CA-125, p53, CD31, |
| | | CA 125, DF 3, VEGFR (*) (ξ) |
| Colon/Bowel | p53, TS, CD43, CEA, PCNA | p53, TS, CD43, CEA, PCNA, MDR-1, |
| | | CD31, CA 242, c-myc, PDECGF, VIP |
| Endometrial | ER/PR, Ki-67, p53, MDR-1 | ER/PR, Ki-67, p53, MDR-1, CD31, |
| | | CA-125, MPR, TSP, ras (ξ) (∞) |
| Lung | p53, LRP, NSE, MDR-1 CEA, CA-125 | p53, LRP, NSE, MDR-1 CEA, CA-125, |
| | | bcl-2, Cyfra 21-1, CA19-9, |
| | | MGMT, MRP (**) (ξ) (ψ) |
| Melanoma | MDR-1, p53, CD31, HMB-45, | MDR-1, p53, CD31, HMB-45, MRP, |
| | MRP, EGFR, Involucrin | EGFR, Involucrin, Bcl-2, c-myc, |
| | | PCNA, Ki67, NIKI |
| | | (ψ) (λ) |
| Oral | p53, MDR-1, MRP, EGFR, PCNA, | p53, MDR-1, MRP, EGFR, PCNA, |
| | CA-125 | CA-125 |
| Peritoneal | CA 19.9, Gastrin, S-100, PCNA, | CA 19.9, Gastrin, S-100, PCNA, NSE, |
| | NSE | MDR, MRP, Ki-67, p53, EGFR |
| Prostrate | AR, HPAP, PSMA, c-erb-2, Ki-67, | AR, HPAP, PSMA, c-erb-2, Ki-67, |
| | GRP | GRP, p53, MDR-1, P-cadherin, |
| | | VEGF, CD31 (π) |
| Sarcoma | p53, MDR-1, MRP, EGFR, O13 | p53, MDR-1, MRP, EGFR, O13, |
| | | VEGR, Bcl-2, c-myc, PCNA, Ki-67 |
| | | (ψ) |
| Stomach [Omentum] | CA 19.9, Gastrin, PP, PCNA, MDR-1, | CA 19.9, Gastrin, PP, PCNA, MDR-1, |
| | S-100, HBP-P | S-100, HBP-P, NSE, LMW Keratin, |
| | | Villin |
| Thyroid | Iodinc-R, Thyro-R, TSH-R, PCNA, | Iodine-R, Thyro-R, TSH-R, PCNA, |
| | p53 | p53, PTH-R, MDR-1, MRP |
| Unkown | p53, Her2/neu, MDR-1, PCNA, | p53, Her2/neu, MDR-1, PCNA, |
| Primary site | CD31, CA-125 | CD31, CA-125, CD34, Ki-67, MPR, |
| | | LRP, CEA (*) (**) (ξ) (ψ) |

The use of various symbols in the comprehensive profiles is intended to provide the clinician with recommendations regarding additional panels that should be run in conjunction with the comprehensive profiles. These symbols represent various panels listed below in Table 9. The symbols are defined as follows:

(ψ)-Cytogenic panel recommended

(χ)-Carcinoma of Unknown Primary Site panel recommended

(ƒ)-Carcinoma panel recommended

(λ)-Epithelial panel recommended

(π)-Bladder vs. Prostate Carcinoma panel recommended

(♥)-Pituitary panel recommended

(∞)-Neuronal panel recommended

(*)-Growth Factor panel recommended

(ξ)-WBC Infiltration panel recommended

(**)-Oncogene/TSG panel recommended

TABLE 9

| PANEL | BMMs |
|---|---|
| Angiogenesis Panel/Index-1 | CD31, CD34, VEGFR, TSP-1, PDGFR-α chain |
| Angiogenesis Panel/Index-2 | p53, TSP-1, CD31, [Indication for “at risk” occult metastasis] |
| Apoptosis Panel | P53, mdm-2, annexin, bcl-2, bax |
| Carcinoma of Unknown | PCNA, p53, Her-2, MDR, ER/PR/AR |
| Primary Site Panel | |
| Carcinoma of Unknown | Her-2, LRP, MDR, CEA, CA125, CD43 (males = PSMA) |
| Primary Site with Metastasis | |
| to Spine or Bones Panel | |
| Carcinoma vs. Lymphoma | LCA, c-kit/myeloid marker = CD117, Ki-67 |
| Panel | |
| Epithelial Panel | Ber-EP4, B72.3, EGFR, EMA |
| Growth Factor-Receptor Panel | c-erb-2, EGFR, c-erb-1, VEGFR, PDGFR, TGFR -I&II |
| | [amplified-indication growth regulation & uncontrolled cell |
| | proliferation] |
| Heat Shock Protein Panel | HSP-PC96, HSP 70, HSP 90 |
| Hormone Receptor Panel | ER/PR/AR |
| Invasion/Metastasis Panel | ICAM, uPa, Pai-2, Bcl-x, TM |
| Keratin Panel #1 | Keratins #39, 43, 50 |
| Keratin Panel #2 | Keratins #45, 56 |
| Keratin Panel #3 | Keratins #34, 39, 40, 43, 48, 50, 50.6 |
| Keratin Panel #4 | Keratins #39, 40, 43, 48, 50, 50.6 |
| Keratin Panel #5 | Keratins #40-68 |
| Lymph Node & Bone | LK/AE-1, CD31, CD34 |
| Marrow MicroMetastasis | |
| Panel | |
| Lymphoma vs. Carcinoma | LCA, c-kit/myeloid marker = CD117, Ki-67 |
| Panel | |
| Multidrug Resistance Panel | MDR-1, MPR, MGMT |
| #1 | |
| Multidrug Resistance Panel | TS, LRP, Topoisomerase I&II |
| #2 | |
| Neural Panel | CD56, GFAP, Leu7, MBP, NF, NSE, β2-Microglobulin, Syn, |
| | NSE, Ubiguitin |
| Neuroendocrine Panel | PGP 9.5, NSE, Chromogranin A, CEA |
| Neuroendocrine Gastrin Panel | Bombesin, CA 19.9, CD56, Leu7 |
| Occult Metastasis Panel #1 | ICAM, uPA, Pai-2, Bcl-x, TM |
| Occult Metastasis Panel #2 | p53, TSP-1, CD31 |
| Oncogene/Tumor Suppressor | TNFR, TGFR, c-myc, p53, ras |
| Gene Panel #1 | |
| Oncogene/Tumor Suppressor | c-fos, c-jun, c-myc, ras |
| Gene Panel #2 | |
| Pituitary Panel | GH, IGF-I, TSH, Adrenocorticotropin, Prolactin |
| Proliferative Panel/Index | Ki-67, c-crb-2, PCNA |
| T & B Lymphocytes Panel | CD3, CD19/Leu12, CD45RO/A6, Leu17 (T-cells, B-cells, |
| | [Helper, Inducer T-cells], Activated T & B cells) |
| Unconventional Multidrug | p53, bcl-2 |
| Resistance Panel | |
| Undifferentiated Carcinoma | p53, Rb, APC, MCC, simple epithelial cytokeratins and |
| Panel | squamous epithelial cytokeratins |
| Undifferentiated Tumor Panel | Calretinin, mucicarmine, CEA, B72.3 |
| White Blood Cell Count | MCG, CD3, CD19/Leu-12, CD41/GPIIB/IIIA, CD45 |
| Infiltration Panel #1 | (Macrophages, T-cells, B-cells, [platelets, megakaryocytes, |
| | megakaryoblasts], leukocytes) |
| White Blood Cell Count | MCG, CD3/Leu3a&b, CD45, CD14/MO2 (Magrophages, |
| Infiltration Panel #2 | Helper T-cells, [Mature monocytes, granulocytes], Leukocytes) |
| White Blood Cell Count | T & B cells = CD3, CD19/Leu12, CD45RO/A6, Leu17 (T-cells, |
| Infiltration Panel #3 | B-cells, [Helper, Inducer T-cells], Activated T & B cells) |

Interpretation of Assay Results

The final interpretation of the results of the foregoing basic and comprehensive profiles relative to a specific patient with a particular stage of tumor growth and treatment history will be left to the primary oncologist treating the patient. Positive results are indicated by the presence of Class I BMMs above or below basal levels or the detection of any amount of Class II BMMs. Typically, the quantity of up-regulated or down-regulated Class I BMMs and detected Class II BMMs will be the primary interpretative indicators, together with their type.

1. One Class I BMM present at non-basal levels:

In this case, the assay evaluation results may be due to some non-cancer related health issue, such as pregnancy, normal menses, etc. Thus, a patient medical history evaluation is made to identify such issues. If there is no non-cancer related explanation for the assay result, the patient is designated as being possibly precancerous and the Class II BMM results are consulted for cancer process information.

2. Two or more Class I BMMs present at non-basal levels:

If the profile demonstrates positive results for two Class I BMMs or Class II BMMs, there is usually a high risk or entering into an oncogenic state. The patient will be designated as precancerous and intervention, be it chemotherapy and/or radiation, may be necessary to prevent the overt onset of cancer. If the profile demonstrates positive results for three or more Class I BMMs or Class II BMMs, the patient is designated a cancerous. First line chemotherapy and/or radiotherapy is performed. The results of the profile will dictate exactly what chemoregimen/radioregimen to follow based on BMM expression and concentration. In particular, a chemmoregimen can be based on selecting a suite of BMM modulating drugs, such as those described above, that are designed to target cells expressing nonbasal levels of Class I BMMs. The drugs will cap the Class I BMMs in such cells. A radioregimen can be based on tumor size and type as determined by the Class II BMMs.

Once a precancerous or cancerous patient has been treated, evaluation of BMM profiles will continue to be monitored to determine if treatment modalities have been efficacious by up-regulation and down-regulation of the BMMs that were initially detected. Additional and possibly modified treatments may then follow.

Accordingly, a cancer comprehensive assay kit for evaluating cancer protein patterns is described herein. Unlike conventional cancer diagnosis, the inventive assay kit is not based on staging. It does not matter what stage the patient's tumor is in or what type it is. An overt objective of the assay is that in the future, stage 2, stage 3 or stage 4 treatment may become a thing of the past because tumors will be neutralized fast enough and early enough, thereby preventing growth progression. A further advantage of the disclosed assay is that a clinician can homogenate the tumor, liquefy it, reduce its size, and dilute it out. Large tumor segments are not required. A tumor can be evaluated in totality. Moreover, serum/plasma specimens can be evaluated, thereby allowing the monitoring the patient's health status. By implementing a series of assay kit evaluations, the clinician may detect remission, recurrence, relapse and metastases. This will, in effect, indicate whether the patient's therapy is effective and allow the clinician to quickly react.

While various embodiments of the invention have been shown and described, it should be apparent that many variations and alternative embodiments could be implemented in accordance with the invention. It is understood, therefore, that the invention is not to be in any way limited except in accordance with the spirit of the appended claims and their equivalents.

I claim:

1. An assay kit for characterizing a cancer tumor for medical diagnosis and treatment, comprising:
a frame structure;
a plurality of test wells associated with said frame structure;
said test wells being arranged to form plural test well rows and plural test well columns;
each test well having a surface configuration adapted to carry a capture protein;
capture proteins coated on said surface configurations of said test wells;
said capture proteins being specific to multiple biomolecular markers (BMMs) and being arranged such that capture proteins specific to a particular BMM are associated with a single test well column;
a set of detection proteins, each of said detection proteins being for use with one of said test well columns and being specific to the same BMM as said capture protein associated with said test well column;
at least some of said test wells of each test well column being adapted to receive assay evaluation samples obtained from a patient tumor sample or from a patient serum/plasma sample; and
said BMMs to which said capture proteins and said detection proteins are specific being selected so that said test well columns may be used to collectively test for a cancer protein pattern based on detected levels of multiple biomolecular markers (BMMs) associated with a patient's tumor, and so that a cancer therapy regimen may be selected based on said cancer protein pattern for eradicating the tumor.

2. An assay kit in accordance with claim 1 wherein said capture proteins and detection proteins are antibodies.

3. An assay kit in accordance with claim 1 wherein said capture proteins and detection proteins are antigens.

4. An assay kit in accordance with claim 1 wherein said test well columns comprise interconnected ones of said test wells so as to be adapted for removal from or addition to said frame structure as a group.

5. An assay kit in accordance with claim 1 wherein some of said test well rows contain BMM samples at predetermined concentrations for establishing standard curves.

6. An assay kit in accordance with claim 1 wherein some of said test well rows contain BMM control samples for establishing assay test controls.

7. An assay kit in accordance with claim 1 wherein there are four to eight test well columns for testing four to eight BMMs as part of a basic test profile.

8. An assay kit in accordance with claim 1 wherein there are more than eight test well columns for testing more than eight BMMs as part of a comprehensive test profile.

9. An assay kit in accordance with claim 1 wherein said BMMs include proteins that can be modulated by protein modulating drugs and said cancer therapy regimen includes protein modulating drugs corresponding to one or more of said BMMs.

10. An assay kit in accordance with claim 1 wherein said BMMs include Class I BMMs representing either tumor promoting or tumor suppressor proteins and Class II BMMs representing tumor marker proteins that provide information about cancer progression.

11. An assay kit for medical diagnosis and treatment of cancer, comprising:
a plurality of test wells;
means for supporting said test wells to define a test well array comprising plural test well rows and plural test well columns;
capture means in said test wells for capturing a protein of interest;
said capture means being specific to multiple biomolecular markers (BMMs) and, being arranged such that capture means specific to a particular BMM are associated with a single test well column;
detection means for binding to said proteins of interest, each of said detection means being for use with one of said test well columns and being specific to the same BMM as said capture means associated with said test well column;
at least some of said test wells of each test well column being adapted to receive assay evaluation samples obtained from a patient tumor sample or from a patient serum/plasma sample; and
said BMMs to which said capture means and said detection means are specific being selected so that said test well columns may be used to collectively test for a cancer protein pattern based on detected levels of multiple biomolecular markers (BMMs) associated with a patient's tumor, and so that a cancer therapy regimen may be selected based on said cancer protein pattern for eradicating the tumor.

12. An assay kit in accordance with claim 11 wherein said assay evaluation sample comprises a homogenate of a solid tumor sample obtained from the patient.

13. An assay kit in accordance with claim 11 wherein said assay evaluation sample comprises a blood serum/plasma sample obtained from the patient.

14. An assay kit in accordance with claim 11 wherein said test well columns comprise interconnected ones of said test wells so as to be adapted for removal from or addition to said frame structure as a group.

15. An assay kit in accordance with claim 11 wherein some of said test well rows contain BMM samples at predetermined concentrations for establishing standard curves.

16. An assay kit in accordance with claim 11 wherein some of said test well rows contain BMM control samples for establishing assay test controls.

17. An assay kit in accordance with claim 11 wherein there are four to eight test well columns for testing four to eight BMMs as part of a basic test profile.

18. An assay kit in accordance with claim 11 wherein there are more than eight test well columns for testing more than eight BMMs as part of a comprehensive test profile.

19. An assay kit in accordance with claim 1 wherein said BMMs include Class I BMMs representing either tumor promoting or tumor suppressor proteins and Class II BMMs representing tumor marker proteins that provide information about cancer progression.

20. An assay kit for characterizing a cancer tumor for medical diagnosis and treatment, comprising:
a frame structure;
a plurality of test wells associated with said frame structure;
said test wells being arranged to form plural test well rows and plural test well columns;
said test well columns comprising interconnected ones of said test wells so as to be adapted for removal from or addition to said frame structure as a group;
each test well having a surface configuration adapted to carry a capture protein;
capture proteins coated on said surface configurations of said test wells;
said capture proteins being specific to multiple biomolecular markers (BMMs) and being arranged such that capture proteins specific to a particular, BMM are associated with a single test well column;
a set of detection proteins, each of said detection proteins being for use with one of said test well columns and being specific to the same BMM as said capture protein associated with said test well column;
at least some of said test wells of each test well column being adapted to receive assay evaluation samples obtained from a patient tumor sample or from a patient serum/plasma sample;
said BMMs to which said capture proteins and said detection proteins are specific being selected so that said test well columns may be used to collectively test for a cancer protein pattern based on detected levels of multiple biomolecular markers (BMMS) associated with a patient's tumor, and so that a cancer therapy regimen may be selected based on said cancer protein pattern for eradicating the tumor;
at least some of said test well rows containing BMM samples at predetermined concentrations for establishing standard curves;
at least some of said test well rows containing BMM control samples for establishing assay test controls;
said BMMs including Class I BMMs representing either tumor promoting or tumor suppressor proteins and Class II BMMs representing tumor marker proteins that provide information about cancer progression.

21. An assay kit for characterizing a cancer tumor for medical diagnosis and treatment, comprising:
a frame structure;
a plurality of test wells associated with said frame structure;
said test wells being arranged to form plural test well rows and plural test well columns;
each test well having a surface configuration adapted to carry a capture protein;
capture proteins coated on said surface configurations of said test wells;
said capture proteins being specific to multiple biomolecular markers (BMMs) and being arranged such that capture proteins specific to a particular BMM are associated with a single test well column;
a set of detection proteins, each of said detection proteins being for use with one of said test well columns and being specific to the same BMM as said capture protein associated with said test well column;
at least some of said test wells of each test well column being adapted to receive assay evaluation samples obtained from a patient tumor sample or from a patient serum/plasma sample;
said BMMs to which said capture proteins and said detection proteins are specific being selected so that said test well columns may be used to collectively test for a cancer protein pattern based on detected levels of multiple biomolecular markers (BMMs) associated with a patient's tumor, and so that a cancer therapy regimen may be selected based on said cancer protein pattern for eradicating the tumor; and
said assay kit being specific to one or more particular cancer types and implemented as either a basic profile comprising a first set of BMMs or a comprehensive profile comprising said first set of BMMs and a second set of BMMs.

22. An assay kit in accordance with claim 21 wherein said assay kit is implemented as a basic ovarian profile with said first set of BMMs comprising ER/PR, Her2/neu, MRP, LRP and EGFR.

23. An assay kit in accordance with claim 21 wherein said assay kit is implemented as a comprehensive ovarian profile with said first and second sets of BMMs comprising ER/PR/AR, Her2/neu, MRP, LRP, EGFR, CA-125, CU-18, PCNA, DF 3, uPA.

24. An assay kit in accordance with claim 21 wherein said assay kit is implemented as a basic ovarian/peritoneal profile with said first set of BMMs comprising S-100, PCNA, MDR-1, EGFR, ER/PR/AR.

25. An assay kit in accordance with claim 21 wherein said assay kit is implemented as a comprehensive ovarian/peritoneal profile with said first and second sets of BMMs comprising S-100, PCNA, MDR-1, EGFR, ER/PR/AR, Ki-67, p53, Her2/neu, MRP, LRP, EGFR, CA-125, uPA.

26. An assay kit in accordance with claim 21 wherein said assay kit is implemented as a basic ovarian/gall bladder/peritoneal profile with said first set of BMMs comprising S-100, PCNA, MDR-1, EGFR ER/PR/AR, PP, p53, c-myc.

27. An assay kit in accordance with claim 21 wherein said assay kit is implemented as a comprehensive ovarian/gall bladder/peritoneal profile with said first and second sets of BMMs comprising S-100, PCNA, MDR-1, EGFR ER/PR/AR, PP, MRP, S-100, NSE, LMW Keratin, p53, TS, CD43, CEA, CD31, CA 242, c-myc, PDECGF, VIP.

28. An assay kit in accordance with claim 21 wherein said assay kit is implemented as a basic ademo-carcinoma profile with said first set of BMMs comprising ACTH, B72.3, BCA225, Bcl-2, CA15.3.

29. An assay kit in accordance with claim 21 wherein said assay kit is implemented as a comprehensive ademo-carcinoma profile with said first and second sets of BMMs comprising ACTH, B72.3, BCA225, Bc1-2, CA15.3, CA125, CEA/D-14, CyclinD1, PCNA, Ki-67, MRP, MDR-1.

30. An assay kit in accordance with claim 21 wherein said assay kit is implemented as a basic bladder profile with said first set of BMMs comprising p53, Her2/neu (p 185), PCNA, MDR-1, EGFR.

31. An assay kit in accordance with claim 21 wherein said assay kit is implemented as a comprehensive bladder profile with said first and second sets of BMMs comprising p53, Her2/neu (p1185), PCNA, MDR-1, EGFR, Ki-67, pan-ras, Bcl-2, Bcl-x, Rb.

32. An assay kit in accordance with claim 21 wherein said assay kit is implemented as a basic brain profile with said first set of BMMs comprising p53, Her2/neu, MGMT, Ki-67, MDR-1, GFAP Syn.

33. An assay kit in accordance with claim 21 wherein said assay kit is implemented as a comprehensive brain profile with said first and second sets of BMMs comprising p53, Her2/neu, MGMT, Ki-67, MDR-1, GFAP, Syn, CD35, CD31 PCNA, VEGFR, PDGFR.

34. An assay kit in accordance with claim 21 wherein said assay kit is implemented as a basic breast profile with said first set of BMMs comprising ER/PR, Her2/neu, TS, BCA-125, MDR-1, MRP.

35. An assay kit in accordance with claim 21 wherein said assay kit is implemented as a comprehensive breast profile with said first and second sets of BMMs comprising ER/PR, Her2/neu, TS, BCA-125, MDR-1, MRP, CA-125, p53, CD31, CA 125, DF 3, VEGFR.

36. An assay kit in accordance with claim 21 wherein said assay kit is implemented as a basic colon/bowel profile with said first set of BMMs comprising p53, TS, CD43, CEA, PCNA.

37. An assay kit in accordance with claim 21 wherein said assay kit is implemented as a comprehensive colon/bowel profile with said first and second sets of BMMs comprising p53, TS, CD43, CEA, PCNA, MDR-1, CD31, CA 242, c-myc, PDECGF, VIP.

38. An assay kit in accordance with claim 21 wherein said assay kit is implemented as a basic endometrial profile with said first set of BMMs comprising ER/PR, Ki-67, p53, MDR-1.

39. An assay kit in accordance with claim 21 wherein said assay kit is implemented as a comprehensive endometrial profile with said first and second sets of BMMs comprising ER/PR, Ki-67, p53, MDR-1, CD31, CA-125, MPR, TSP, ras.

40. An assay kit in accordance with claim 21 wherein said assay kit is implemented as a basic lung profile with said first set of BMMs comprising p53, LRP, NSE, MDR-1 CEA, CA-125.

41. An assay kit in accordance with claim 21 wherein said assay kit is implemented as a comprehensive lung profile with said first and second sets of BMMs comprising p53, LRP, NSE, MDR-1 CEA, CA-125, bcl-2, Cyfra 21-1, CA 19-9, MGMT, MRP.

42. An assay kit in accordance with claim 21 wherein said assay kit is implemented as a basic melanoma profile with said first set of BMMs comprising MDR-1, p53, CD31, HMB-45, MRP, EGFR, Involucrin.

43. An assay kit in accordance with claim 21 wherein said assay kit is implemented as a comprehensive melanoma profile with said first and second sets of BMMs comprising MDR-1, p53, CD31, HMB-45, MRP, EGFR, Involucnin, Bcl-2, c-myc, PCNA, Ki67, NIKI.

44. An assay kit in accordance with claim 21 wherein said assay kit is implemented as a basic oral profile with said first set of BMMs comprising p53, NMR-1, MRP, EGFR, PCNA, CA-125.

45. An assay kit in accordance with claim 21 wherein said assay kit is implemented as a comprehensive oral profile with said first and second sets of BMMs comprising p53, MDR-1, MRP, EGFR, PCNA, CA-125.

46. An assay kit in accordance with claim 21 wherein said assay kit is implemented as a basic peritoneal profile with said first set of BMMs comprising CA19.9, Gastrin, S-100, PCNA, NSE.

47. An assay kit in accordance with claim 21 wherein said assay kit is implemented as a comprehensive peritoneal profile with said first and second sets of BMMs comprising CA19.9, Gastrin, S-100, PCNA, NSE, MDR, MRP, Ki-67, p53, EGFR.

48. An assay kit in accordance with claim 21 wherein said assay kit is implemented as a basic prostrate profile with said first set of BMMs comprising AR, HPAP, PSMA, c-erb-2, Ki-67, GRP.

49. An assay kit in accordance with claim 21 wherein said assay kit is implemented as a comprehensive prostrate profile with said first and second sets of BMMs comprising AR, HPAP, PSMA, c-erb-2, Ki-67, GRP, p53, MDR-1, P-cadherin, VEGF, CD31.

50. An assay kit in accordance with claim 21 wherein said assay kit is implemented as a basic sarcoma profile with said first set of BMMs comprising p53, MDR-1, MRP, EGFR, O13.

51. An assay kit in accordance with claim 21 wherein said assay kit is implemented as a comprehensive sarcoma profile with said first and second sets of BMMs comprising p53, MDR-1, MRP, EGFR, O13, VEGR, Bc1-2, c-myc, PCNA, Ki-67.

52. An assay kit in accordance with claim 21 wherein said assay kit is implemented as a basic stomach profile with said first set of BMMs comprising CA19.9, Gastrin, PP, PCNA, MDR-1, S-100, HBP-P.

53. An assay kit in accordance with claim 21 wherein said assay kit is implemented as a comprehensive stomach profile with said first and second sets of BMMs comprising CA19.9, Gastrin, PP, PCNA, MDR-1, S-100, HBP-P, NSE, LMW Keratin, Villin.

54. An assay kit in accordance with claim 21 wherein said assay kit is implemented as a basic thyroid profile with said first set of BMMs comprising Iodine-R, Thyro-R, TSH-R, PCNA, p53.

55. An assay kit in accordance with claim 21 wherein said assay kit is implemented as a comprehensive thyroid profile with said first and second sets of BMMs comprising Iodine-R, Thylo-R, TSH-R, PCNA, p53, PTH-R, MDR-1, MRP.

56. An assay kit in accordance with claim 21 wherein said assay kit is implemented as a basic unknown primary site profile with said first set of BMMs comprising p53, Her2/neu, MDR-1, PCNA, CD31, CA-125.

57. An assay kit in accordance with claim 21 wherein said assay kit is implemented as a comprehensive unknown primary site profile with said first and second sets of BMMs comprising p53, Her2/neu, MDR-1, PCNA, CD31, CA-125, CD34, Ki-67, MPR, LRP, CEA.

58. An assay kit for characterizing a cancer tumor for medical diagnosis and treatment, comprising:
a frame structure;
a plurality of test wells associated with said frame structure;
said test wells being arranged to form plural test well rows and plural test well columns;
each test well having a surface configuration-adapted to carry a capture protein;
capture proteins coated on said surface configurations of said test wells;
said capture proteins being specific to multiple biomolecular markers (BMMs) and being arranged such that capture proteins specific to a particular BMM are associated with a single test well column;
a set of detection proteins, each of said detection proteins being for use with one of said test well columns and being specific to the same BMM as said capture protein associated with said test well column;
at least some of said test wells of each test well column being adapted to receive assay evaluation samples obtained from a patient tumor sample or from a patient serum/plasma sample;
said BMMs to which said capture proteins and said detection proteins are specific being selected so that said test well columns may be used to collectively test for a cancer protein pattern based on detected levels of multiple biomolecular markers (BMMs) associated with a patient's tumor, and so that a cancer therapy regimen may be selected based on said cancer protein pattern for eradicating the tumor; and
said assay kit being implemented as a panel comprising a set of BMMs selected to provide cancer diagnostic information.

59. An assay kit in accordance with claim 58 wherein said assay kit is implemented as an angiogenesis panel with said BMMs comprising CD31, CD34, VEGFR, TSP-1, PDGFR-α chain.

60. An assay kit in accordance with claim 58 wherein said assay kit is implemented as an angiogenesis panel with said BMMs comprising p53, TSP-1, CD31.

61. An assay kit in accordance with claim 58 wherein said assay kit is implemented as an apoptosis panel with said BMMs comprising P53, mdm-2, annexin, bcl-2, bax.

62. An assay kit in accordance with claim 58 wherein said assay kit is implemented as an apoptosis panel with said BMMs comprising P53, mdm-2, annexin, bcl-2, bax.

63. An assay kit in accordance with claim 58 wherein said assay kit is implemented as a carcinoma of unknown site panel with said BMMs comprising PCNA, p53, Her-2, MDR, ER/PR/AR.

64. An assay kit in accordance with claim 58 wherein said assay kit is implemented as a carcinoma of unknown site with metastasis to spine or bones panel with said BMMs comprising Her-2, LRP, MDR, CEA, CA125, CD43, PSMA.

65. An assay kit in accordance with claim 58 wherein said assay kit is implemented as a carcinoma vs. Lymphoma panel with said BMMs comprising LCA, c-kit/myeloid marker CD117, Ki-67.

66. An assay kit in accordance with claim 58 wherein said assay kit is implemented as an epithelial panel with said BMMs comprising Ber-EP4, B72.3, EGFR, EMA.

67. An assay kit in accordance with claim 58 wherein said assay kit is implemented as a growth factor receptor panel with said BMMs comprising c-erb-2, EGFR, c-erb-1, VEGFR, PDGFR, TGFR-I&II.

68. An assay kit in accordance with claim 58 wherein said assay kit is implemented as a heat shock protein panel with said BMMs comprising HSP-PC96, HSP 70, HSP 90.

69. An assay kit in accordance with claim 58 wherein said assay kit is implemented as a hormone receptor panel with said BMMs comprising ER/PR/AR.

70. An assay kit in accordance with claim 58 wherein said assay kit is implemented as an invasion metastasis panel with said BMMs comprising ICAM, uPa, Pai-2, Bcl-x, TM.

71. An assay kit in accordance with claim 58 wherein said assay kit is implemented as a keratin panel with said BMMs comprising Keratins #39, 43, 50.

72. An assay kit in accordance with claim 58 wherein said assay kit is implemented as a keratin panel with said BMMs comprising Keratins #45, 56.

73. An assay kit in accordance with claim 58 wherein said: assay kit is implemented as a keratin panel with said BMMs comprising Keratins #34, 39, 40, 43, 48, 50, 50.6.

74. An assay kit in accordance with claim 58 wherein said assay kit is implemented as a keratin panel with said BMMs comprising Keratins #40-68.

75. An assay kit in accordance with claim 58 wherein said assay kit is implemented as a lymph node and bone marrow micrometastasis panel with said BMMs comprising LK/AE-1, CD31, CD34.

76. An assay kit in accordance with claim 58 wherein said assay kit is implemented as a lymphoma versus carcinoma panel with said BMMs comprising LCA, c-kit/myeloid marker=CD117, Ki-67.

77. An assay kit in accordance with claim 58 wherein said assay kit is implemented as a multidrug resistance panel with said BMMs comprising MDR-1, MPR, MGMT.

78. An assay kit in accordance with claim 58 wherein said assay kit is implemented as a multidrug resistance panel with said BMMs comprising TS, LRP, Topoisomerase I&II.

79. An assay kit in accordance with claim 58 wherein said assay kit is implemented as a neural panel with said BMMs comprising CD56, GFAP, Leu7, MBP, NF, NSE, β2-Microglobulin, Syn, NSE, Ubiguitin.

80. An assay kit in accordance with claim 58 wherein said assay kit is implemented as a neuroendocrine panel with said BMMs comprising PGP 9.5, NSE, Chromogranin A, CEA.

81. An assay kit in accordance with claim 58 wherein said assay kit is implemented as a neuroendocrine gastrin panel with said BMMs comprising Bombesin, CA19.9, CD56, Leu7.

82. An assay kit in accordance with claim 58 wherein said assay kit is implemented as an occult metastasis panel with said BMMs comprising ICAM, uPA, Pai-2, Bcl-x, TM.

83. An assay kit in accordance with claim 58 wherein said assay kit is implemented as an occult metastasis panel with said BMMs comprising p53, TSP-1, CD31.

84. An assay kit in accordance with claim 58 wherein said assay kit is implemented as an oncogene/tumor suppressor gene panel with said BMMs comprising TNFR, TGFR, c-myc, p53, ras.

85. An assay kit in accordance with claim 58 wherein said assay kit is implemented as an oncogenene/tumor suppressor gene panel with said BMMs comprising c-fos, c-jun, c-myc, ras.

86. An assay kit in accordance with claim 58 wherein said assay kit is implemented as a pituitary panel with said BMMs comprising GH, IGF-I, TSH, Adrenocorticotropin, Prolactin.

87. An assay kit in accordance with claim 58 wherein said assay kit is implemented as a proliferative panel with said BMMs comprising Ki-67, c-erb-2, PCNA.

88. An assay kit in accordance with claim 58 wherein said assay kit is implemented as an T & B lymphocytes panel with said BMMs comprising CD3, CD19/Leu12, CD45RO/A6, Leul7 (T-cells, B-cells, [Helper, Inducer T-cells], Activated T&B cells).

89. An assay kit in accordance with claim 58 wherein said assay kit is implemented as an unconventional multidrug resistance panel with said BMMs comprising p53, bcl-2.

90. An assay kit in accordance with claim 58 wherein said assay kit is implemented as an undifferentiated carcinoma panel with said BMMs comprising p53, Rb, APC, MCC, simple epithelial cytokeratins and squamous epithelial cytokeratins.

91. An assay kit in accordance with claim 58 wherein said assay kit is implemented as an undifferentiated tumor panel with said BMMs comprising calretinin, mucicarmine, CEA, B72.3.

92. An assay kit in accordance with claim 58 wherein said assay kit is implemented as a white blood cell count panel with said BMMs comprising MCG, CD3, CD19/Leu-12, CD41/GPIIB/IIIA, CD45 (Macrophages, T-cells, B-cells, [platelets, megakaryocytes, megakaryoblasts], leukocytes).

93. An assay kit in accordance with claim 58 wherein said assay kit is implemented as a white blood cell count panel with said BMMs comprising MCG, CD3/Leu3a&b, CD45, CD14/M02 (Magrophages, HelperT-cells, [Mature monocytes, granulocytes], Leukocytes).

94. An assay kit in accordance with claim 58 wherein said assay kit is implemented as a white blood cell count panel with said BMMs comprising T&B cells=CD3, CD19/Leul2, CD45RO/A6, Leu17 (T-cells, B-cells, [Helper, Inducer T-cells], Activated T&B cells).
